(19) United States
(12) Patent Application Publication
(10) Pub. No.: US 2026/0053960 A1
(43) Pub. Date: Feb. 26, 2026

(54) DIMERIC RADIOPHARMACEUTICALS, COMPOSITIONS THEREOF AND USES THEREOF

(71) Applicant: Clarity Pharmaceuticals Limited, Eveleigh, New South Wales (AU)

(72) Inventors: Matthew John HARRIS, Eveleigh (AU); Ellen Marianne VAN DAM, Eveleigh (AU); Lachlan Eion MCINNES, Eveleigh (AU)

(21) Appl. No.: 19/102,741

(22) PCT Filed: Aug. 11, 2023
(86) PCT No.: PCT/AU2023/050761

§ 371 Date: Feb. 10, 2025

(30) Foreign Application Priority Data
Aug. 11, 2022 (AU) 2022902267

(51) Int. Cl. A61K 51/08; A61K 51/04

(52) U.S. Cl. CPC A61K 51/088; A61K 51/0482; A61K 51/083; A61K 2123/00

(57) ABSTRACT

The present invention relates to compounds comprising a metal chelator and two fragments capable of binding to type 2 somatostatin receptors, compositions thereof and uses thereof in methods of treatment.

### FIELD OF THE INVENTION

The present invention relates to compounds for use as radiopharmaceuticals comprising a metal chelator and fragments that are capable of binding to somatostatin receptors. The present invention also relates to compositions thereof and uses thereof in methods of radioimaging and treatment of cancer.

### BACKGROUND

Type 2 somatostatin receptors (SSTR2) are G-protein coupled receptors that are expressed on the surface of some cancerous tumours. Binding at these receptors may inhibit growth of the tumour, while delivery of internal beam radiation by exposure of the tumour to a radiation source is thought to further inhibit growth of the tumour. While SSTR2 may be a target for the purposes of treatment of a cancer, selective targeting often presents difficulties as the receptor is also expressed in other tissues and organ systems. Furthermore, even if selective targeting of the SSTR2 on tumour cells is achieved, the ligand binding to the receptor must also have sufficient retention at the tumour site and the appropriate physical properties.

Other difficulties encountered when designing and producing radiopharmaceuticals include low compound yields during synthesis and radiolabelling, limited or reduced radiochemical stability of the compounds when coordinated with a radioisotope and general solubility and stability of the compound under both in vitro and in vivo conditions. Since the compounds are intended to coordinate a radioisotope, they must also display sufficient radiolytic stability for a time, for example, for at least one half-life of the desired radioisotope. Where the compounds do not have the requisite radiolytic stability, unwanted toxicity and off-target damage to healthy tissue can occur. While one option for treatment and/or radioimaging of a cancer that is known to express SSTR2 is to administer known compounds at a higher dose to increase efficacy of treatment or resolution of images, this may lead to exacerbation of the unwanted side effects related to the administered compound.

There remains a need for radiopharmaceuticals that show greater retention at the tumour sites for the purposes of both radioimaging and treatment of the cancer, while limiting toxicity and off-target radiation damage to levels that are within (or lower than) acceptable limits.

### SUMMARY OF THE INVENTION

The present invention relates to compounds for use as radiopharmaceuticals, where the compounds comprise moieties that are capable of binding to somatostatin type 2 receptors (SSTR2), compositions thereof and their use in methods of treatment and radioimaging. The present inventors believe that the compounds disclosed herein comprising moieties capable of binding to SSTR2 and a radioisotope coordinated to a suitable chelator allow for more efficient treatment of cancer in a subject and the ability to obtain images of a higher resolution for the purposes of diagnosis.

Although the compounds disclosed herein contain an additional linker and moiety capable of binding to SSTR2, the present inventors believe that the compounds show greater affinity for tumour sites that express SSTR2 when compared to compounds comprising a single linker and binding group. Without wishing to be bound by theory, the present inventors believe that this may lead to an increased amount of drug retained in the tumour leading to greater inhibition of tumour growth or a reduction in the amount of the radiolabelled compound that is administered to the patient. Since treatment of cancers often require multiple treatment rounds, the administration of a compound that can provide more efficient treatment may lead to fewer rounds of treatment and therefore less burden on the patient.

In contrast to compounds containing a single octreotate moiety, the present inventors have found that compounds disclosed herein show increased binding and retention at sites expressing SSTR2, i.e. a greater proportion of the administered compound is bound and retained at the desired site. This in turn leads to more efficient treatment and imaging of a cancer that is associated with the expression of SSTR2. Since the compounds disclosed herein show better binding to the target site, a smaller dose of the compound can be administered to the subject. As the compounds disclosed herein are capable of chelating a suitable radionuclide, this then means that a smaller dose of radiation can be administered to the subject, while still providing images or treatment of a requisite standard. Where a smaller dose of radiation is administered, this reduces the likelihood and/or severity of any unwanted side effects that arise due to the administration of a radionuclide, i.e. off-target radiation damage.

Since the compounds of the present invention contain a radionuclide for radioimaging and radiotherapy, the compounds must be retained for a sufficient time for imaging or therapy, however the compounds must also be metabolised and excreted from the subject after a time. The present inventors have found that even though the compounds of the invention containing two SSTR2-binding moieties and therefore have a greater molecular weight, the compounds show improved binding and retention so as to provide better images during radioimaging and better therapy during radiotherapy due to retention at the cancer site expressing SSTR2, the compounds also show the requisite stability and physical properties for metabolism in an acceptable timeframe. Nonetheless, a person skilled in the art would understand that a larger compound (and thus a compound having a greater molecular weight) would not necessarily provide a comparable therapeutic effect, even if the same targeting groups are used. This may be because the increase in size and molecular weight may adversely impact physical properties of the compound, such as solubility at a given pH. Where solubility of the compound is lowered, this then results in a reduced therapeutic effect (when compared to a similar compound having a single linker/SSTR2 binding moiety) since less of the administered compound reaches the target site. As seen in FIGS. 1 and 2, the compounds of the present invention having two peptide moieties and therefore a greater molecular weight show binding to a tumor expressing SSTR2. Without wishing to be bound by theory, the present inventors believe that even though the compounds disclosed herein are larger and have a greater molecular weight, the compounds are still able to show requisite binding to a tumour site expressing SSTR2 and are therefore useful in the treatment and/or radioimaging of a cancer associated with expression of SSTR2. Furthermore, the compounds of the present invention not only bind to SSTR2, but are also retained for a time such that the therapeutic effect intended can be delivered to the target tumor site.

The compounds of the present invention comprise a single sarcophagine and two linker moieties bound to two moieties that show affinity for binding at SSTR2, where the octreotate moieties show affinity for binding at SSTR2. The sarcophagine component is capable of chelating a copper radioisotope, which are known to be useful in radioimaging and radiotherapy. Known drawbacks of radioisotope-based imaging and therapy is that loss of the radioisotope from the chelator can cause radiation damage to healthy tissue, since the unchelated radioisotope can circulate freely. Furthermore, since not all of the administered radioisotope reaches the target site, more of the chelated compound may be required in order to provide the required therapeutic effect.

Existing compounds that are capable of chelating copper compounds for radioimaging and radiotherapy include those with a DOTA or NOTA macrocycle, for example. The present inventors believe that the compounds disclosed herein comprising a sarcophagine allow for improved methods of treatment, as the sarcophagine chelator as used herein better retains the copper radioisotope under both in vitro and in vivo conditions when compared to compounds having a different chelator. The present inventors believe that this advantage relates to the ability of the sarcophagine to better withstand the products of radioactive decay from the copper radioisotopes, such that radiolysis of the compounds is generally reduced.

In a first aspect, the present invention provides a compound of Formula (I):

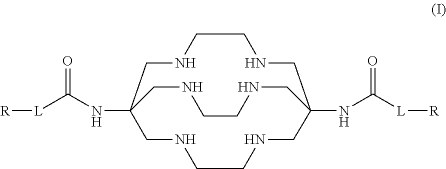

or a pharmaceutically acceptable salt, complex, isomer, solvate or prodrug thereof,
wherein
each R is a moiety that binds to SSTR2; and
each -L- is a linker moiety conjugated to the moieties that binds to SSTR2 and to the sarcophagine.

In a second aspect, the present invention provides a compound of Formula (II):

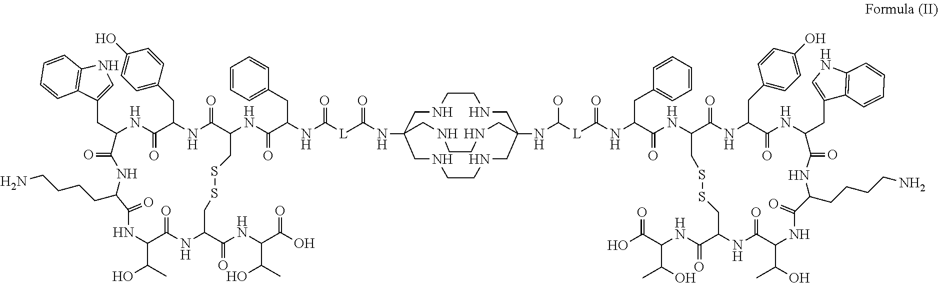

or a pharmaceutically acceptable salt, complex, isomer, solvate or prodrug thereof,
wherein
each -L- is a linker moiety conjugated to the moieties that binds to SSTR2 and to the sarcophagine.

In some embodiments each linker group “-L-” is independently an optionally substituted —C₁-C₁₀alkylene-, —C₂-C₁₀alkenylene- or —C₂-C₁₀alkynylene-group, one or more amino acids residues, one or more PEG groups, or combinations thereof; wherein one or more of the carbon atoms in the alkylene, alkenylene or alkynylene group may be replaced with NH, S, O, a C₅-C₈ aromatic or aliphatic cyclic group or a C₅-C₈ aromatic or aliphatic heterocyclic group.

The compounds of Formulae (I) and (II) comprise a sarcophagine, i.e. a 3,6,10,13,16,19-hexaazabicyclo[6.6.6] icosane that is capable of chelating a metal ion. The sarcophagine of Formula (I) contains six nitrogen atoms, where one or more of the nitrogen atoms may be protected with a suitable protecting group. The compounds also comprise linker groups that attach two octreotate moieties to the sarcophagine in order to provide a compound of Formula (I). Without wishing to be bound by theory, the present inventors believe that each component of the compounds disclosed herein (i.e. the sarcophagine, linker and octreotate) contributes to provide the requisite metabolic and/or radiolytic stability, solubility and flexibility, such that the compound of Formula (I) is useful as a radiopharmaceutical.

In certain embodiments, the compound of Formula (II) has the structure of Formula (IIa):

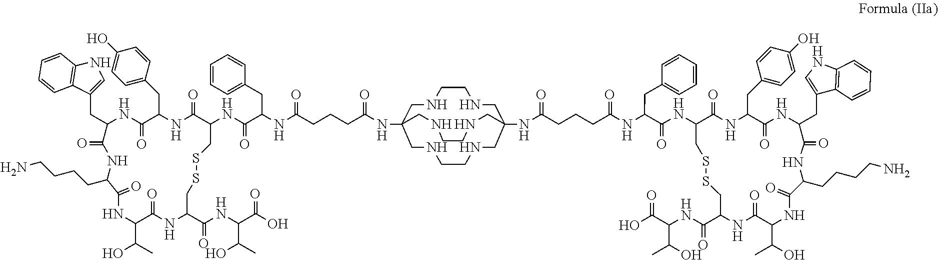 [SMILES: CC(O)C(NC(=O)C1CSSCC(NC(=O)C(Cc2ccccc2)NC(=O)CCCC(=O)NC23CNCCNCC(NC(=O)CCCC(=O)NC(Cc4ccccc4)C(=O)NC4CSSCC(C(=O)NC(C(=O)O)C(C)O)NC(=O)C(C(C)O)NC(=O)C(CCCCN)NC(=O)C(Cc5c[nH]c6ccccc56)NC(=O)C(Cc5ccc(O)cc5)NC4=O)(CNCCNC2)CNCCNC3)C(=O)NC(Cc2ccc(O)cc2)C(=O)NC(Cc2c[nH]c3ccccc23)C(=O)NC(CCCCN)C(=O)NC(C(C)O)C(=O)N1)C(=O)O]

or a pharmaceutically acceptable salt, complex, isomer, solvate or prodrug thereof.

In certain embodiments, the compound of Formula (II) has the structure of Formula (IIb):

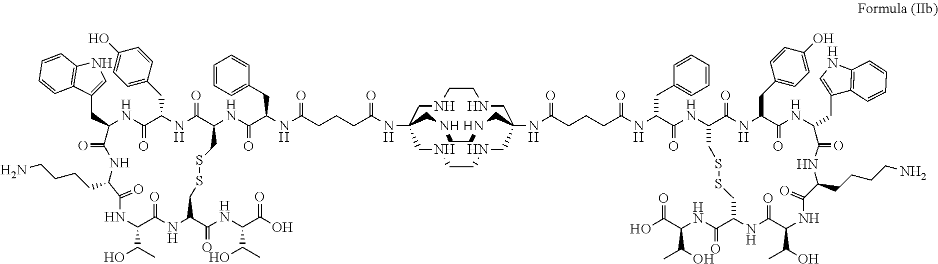

In certain embodiments, the metal chelator in the compound of Formula (I) is complexed with an ion of a metal selected from the group consisting of Cu, Tc, Gd, Ga, In, Co, Re, Fe, Mg, Ag, Rh, Pt, Cr, Ni, V, Ir, Zn, Cd, Mn, Ru, Pd, Hg, Ti, Lu, Sc, Zr, Y, Ac, As, Ra and Pb.

In some embodiments, the metal ion complexed in the metal chelator is a radionuclide.

In some embodiments, the compound of Formula (I) is complexed with a radionuclide selected from the group consisting of ⁶⁰Cu, ⁶¹Cu, ⁶²Cu, ⁶⁴Cu, ⁶⁷Cu, ⁶⁸Ga, ⁹⁰Y, ¹¹¹In, ¹⁷⁷Lu, ¹⁸⁸Re, ²¹¹As, ²¹²Pb and ²²⁵Ac.

In a third aspect, the present invention provides a composition comprising a compound of Formula (I) as defined in the first aspect and one or more pharmaceutically acceptable excipients.

The present inventors believe that the compounds coordinated with a radionuclide may be used as a radiopharmaceutical or radioimaging agent, if the compound can bind sufficiently to the desired site and deliver a radionuclide to the same site for the purposes of imaging or therapy.

In a fourth aspect, the present invention provides a method for radioimaging a cancer in a subject in need thereof, the method comprising administering to the subject a compound of Formula (I) according to the first aspect or a composition according to the second aspect, wherein the compound is coordinated with a radionuclide.

In certain embodiments, the method for radioimaging comprises imaging by positron emission tomography (PET) or single-photon emission computed tomography (SPECT).

In a fifth aspect, the present invention provides a method for the treatment of a cancer in a subject in need thereof, the method comprising administering to the subject a compound of Formula (I) according to the first aspect or a composition according to the second aspect, wherein the compound is coordinated with a radionuclide.

In particular embodiments, the cancer is characterised by expression of somatostatin type 2 receptors (SSTR2).

In a sixth aspect, the present invention provides a use of a compound of Formula (I) according to the first aspect in the manufacture of a medicament for radioimaging a cancer, wherein the compound is coordinated with a radionuclide.

In certain embodiments, the radioimaging comprises imaging by positron emission tomography (PET) or single-photon emission computed tomography (SPECT).

In a seventh aspect, the present invention provides a use of a compound of Formula (I) according to the first aspect in the manufacture of a medicament for treating a cancer, wherein the compound is coordinated with a radionuclide.

In certain embodiments, the cancer is characterised by the expression of somatostatin type 2 receptors (SSTR2).

### BRIEF DESCRIPTION OF THE FIGURES

FIG. 1. Mean±SEM of injected dose (%) per gram of tissue weight (% ID/g) is shown for each tissue after injection of SARbisTATE at 1 h, 4 h, 24 h and 48 h (A); and with a split y-axis (B).

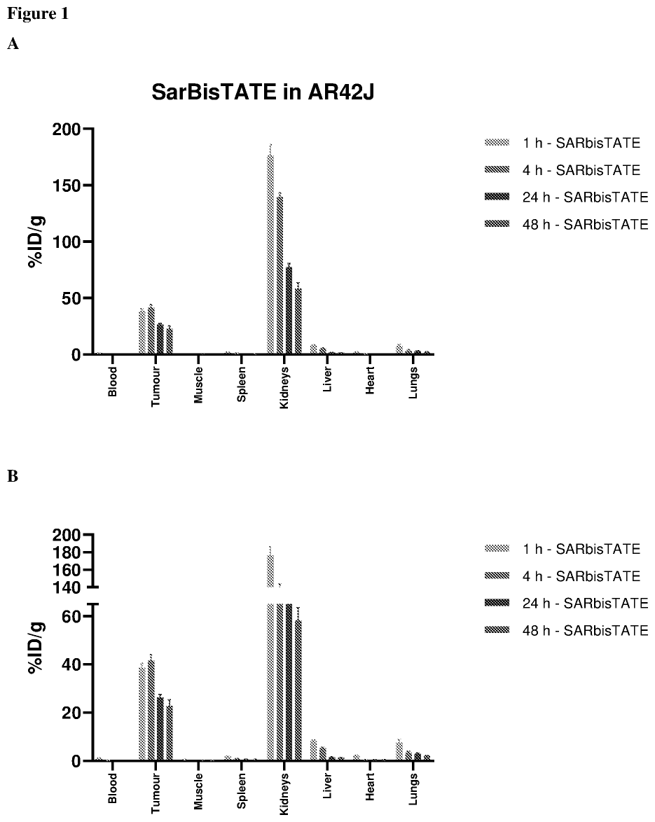

FIG. 2. 5 MBq ⁶⁴Cu-SarbisTATE was administered to 4 subjects and biodistribution of the radiolabelled compound determined after 1 h, 4 h, 24 h and 48 h.

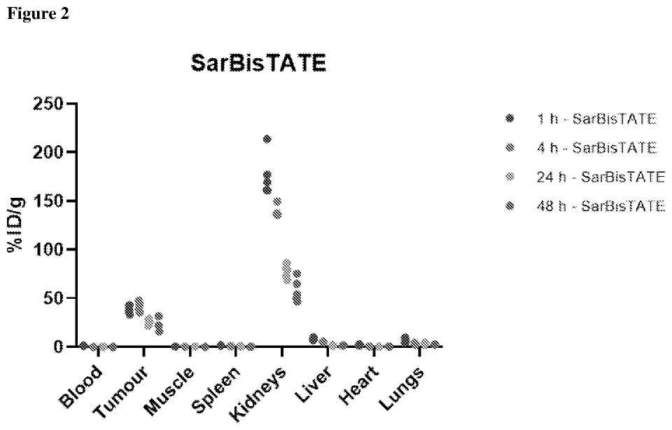

### DETAILED DESCRIPTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The term “about” or “approximately” as used herein means within an acceptable error range for the particular value as determined by one of ordinary skill in the art, which will depend in part on how the value is measured or determined, i.e., the limitations of the measurement system.

Unless defined otherwise, all technical and scientific terms used herein have the same meaning as commonly understood by those of ordinary skill in the art to which the invention belongs. For the purposes of the present invention, the following terms are defined below.

In a first aspect, the present invention provides a compound of Formula (I):

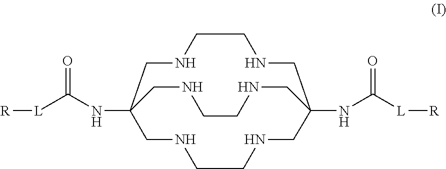

or a pharmaceutically acceptable salt, complex, isomer, solvate or prodrug thereof,
wherein
each R is a moiety that binds to SSTR2; and
each -L- is a linker moiety conjugated to the moieties that binds to SSTR2 and to the sarcophagine.

A number of suitable moieties that bind to SSTR2 are known in the art and include, but are not limited to:

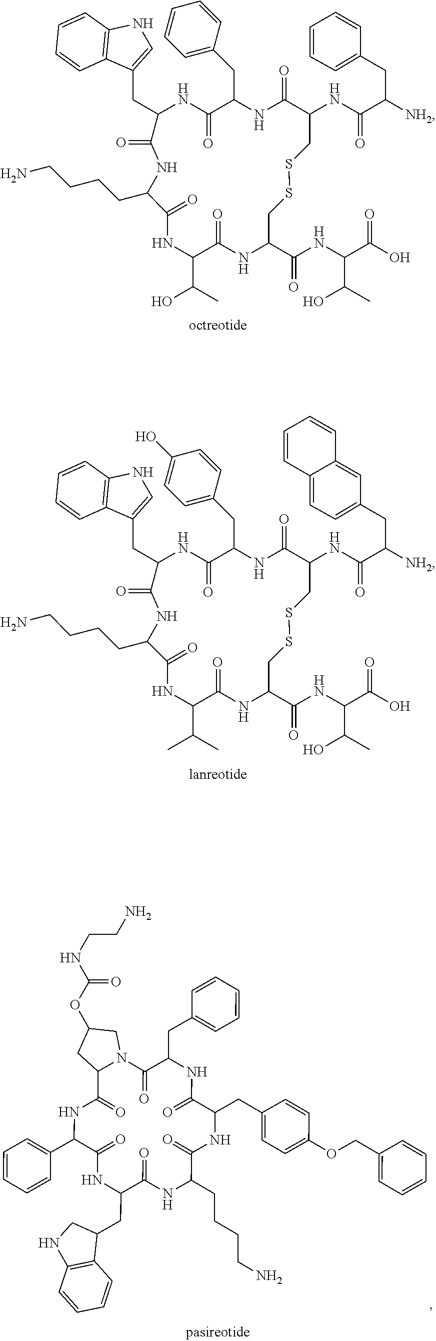 [SMILES: CC(O)C(NC(=O)C1CSSCC(NC(=O)C(N)Cc2ccccc2)C(=O)NC(Cc2ccccc2)C(=O)NC(Cc2c[nH]c3ccccc23)C(=O)NC(CCCCN)C(=O)NC(C(C)O)C(=O)N1)C(=O)O ; CC(C)C1NC(=O)C(CCCCN)NC(=O)C(Cc2c[nH]c3ccccc23)NC(=O)C(Cc2ccc(O)cc2)NC(=O)C(NC(=O)C(N)Cc2ccc3ccccc3c2)CSSCC(C(=O)NC(C(=O)O)C(C)O)NC1=O ; NCCCCC1NC(=O)C(CC2CNc3ccccc32)NC(=O)C(c2ccccc2)NC(=O)C2CC(OC(=O)NCCN)CN2C(=O)C(Cc2ccccc2)NC(=O)C(Cc2ccc(OCc3ccccc3)cc2)NC1=O]

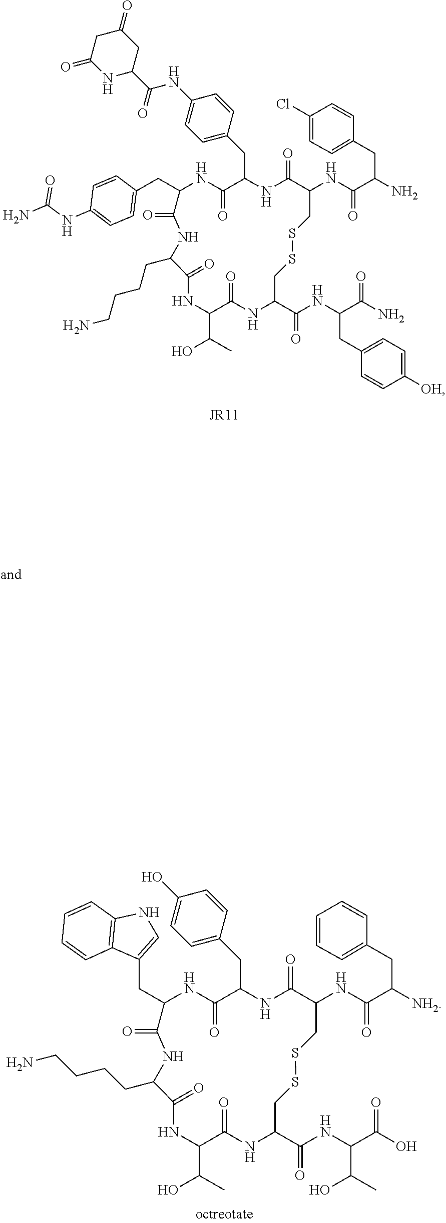 [SMILES: CC(O)C1NC(=O)C(CCCCN)NC(=O)C(Cc2ccc(NC(N)=O)cc2)NC(=O)C(Cc2ccc(NC(=O)C3CC(=O)CC(=O)N3)cc2)NC(=O)C(NC(=O)C(N)Cc2ccc(Cl)cc2)CSSCC(C(=O)NC(Cc2ccc(O)cc2)C(N)=O)NC1=O ; CC(O)C(NC(=O)C1CSSCC(NC(=O)C(N)Cc2ccccc2)C(=O)NC(Cc2ccc(O)cc2)C(=O)NC(Cc2c[nH]c3ccccc23)C(=O)NC(CCCCN)C(=O)NC(C(C)O)C(=O)N1)C(=O)O]

In one embodiment. R is selected octreotide, lanreotide, pasireotide, octreotate and combinations thereof.

In another embodiment, each R is octreotate. Accordingly, in a further aspect the present invention provides a compound of Formula (II):

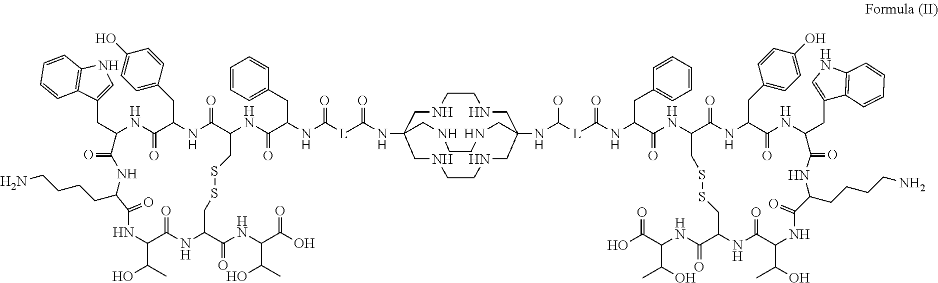

or a pharmaceutically acceptable salt, complex, isomer, solvate or prodrug thereof,
wherein
each -L- is a linker moiety conjugated to the moieties that binds to SSTR2 and to the sarcophagine.

In certain embodiments, the linkers in the compound of Formulae (I) or (II) may be the same or different. In certain embodiments, the linkers in the compound of Formulae (I) or (II) are different. In embodiments, the linkers in the compound of Formulae (I) or (II) are the same. Suitable linkers in include, for instance, N-succinimidyl-4-(2-pyridylthio) propanoate (SPDP), N-succinimidyl-4-(2-pyridylthio) pentanoate (SPP).

In certain embodiments, the linker is of the following formula (III):

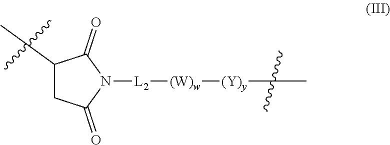

wherein
L₂ is cycloalkylene-carbonyl, (C₂-C₆)alkyl or (C₂-C₆)alkyl-carbonyl;
W is an amino acid unit; w is an integer comprised of 0 to 5;
Y is PAB-carbonyl with PAB being

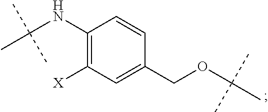

x can be H or

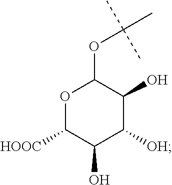

y is 0 or 1; and
the wavy lines indicate the points of attachment to the moiety that binds to SSTR2 and to the sarcophagine.

In an embodiment, L₂ is of the following formula:

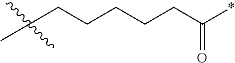 [SMILES: *CCCCCC(*)=O]

wherein
the asterisk indicates the point of attachment to (W)_{w}; and
the wavy line indicates the point of attachment to the nitrogen atom of a maleimide moiety of formula:

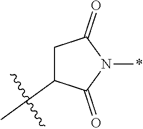 [SMILES: *C1CC(=O)N(*)C1=O]

In an embodiment of the invention, w=0, or w=2 and then (W)w is selected from:

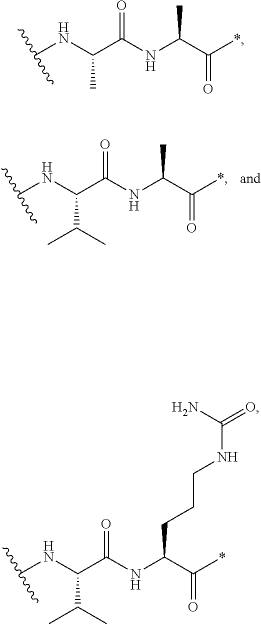

wherein
the asterisk indicates the point of attachment to (Y)_{y}; and
the wavy line indicates the point of attachment to L₂.

In an embodiment of the invention the linker is selected from:

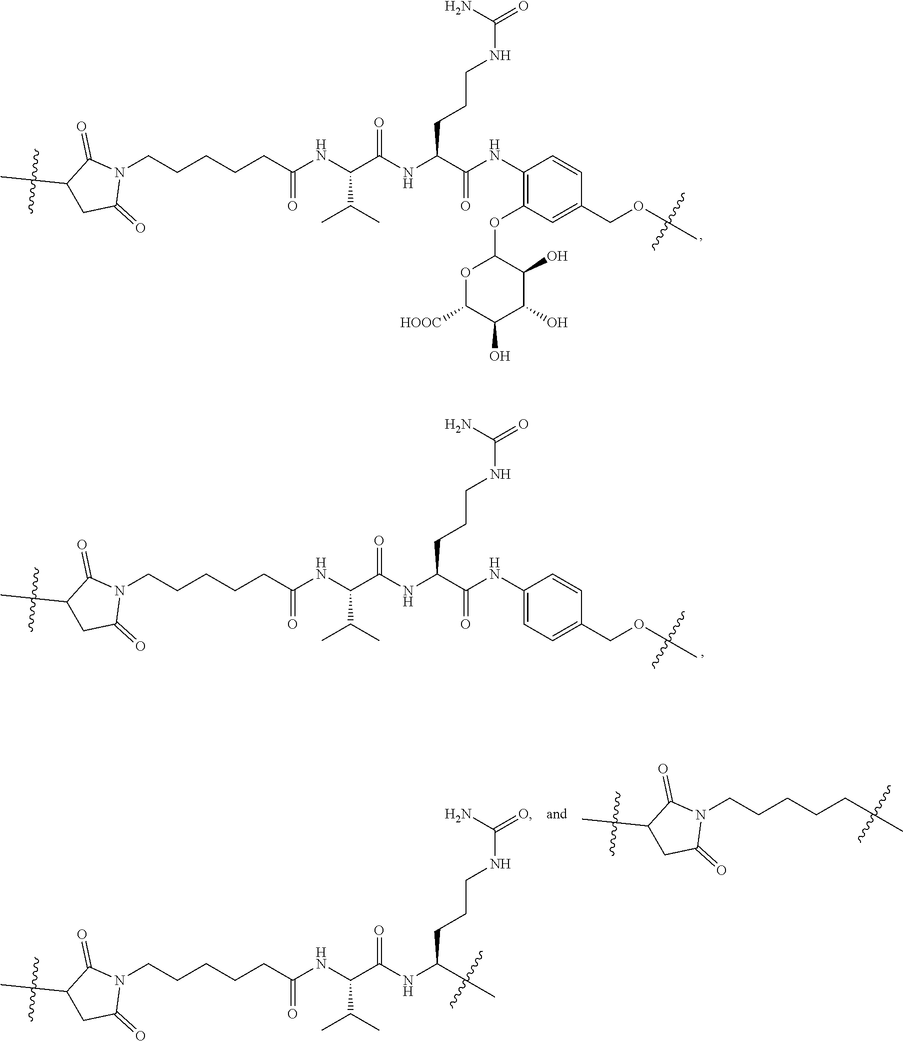 [SMILES: *OCc1ccc(NC(=O)[C@H](CCCNC(N)=O)NC(=O)[C@@H](NC(=O)CCCCCN2C(=O)CC(*)C2=O)C(C)C)c(OC2O[C@@H](C(=O)O)[C@H](O)[C@@H](O)[C@@H]2O)c1 ; *OCc1ccc(NC(=O)[C@H](CCCNC(N)=O)NC(=O)[C@@H](NC(=O)CCCCCN2C(=O)CC(*)C2=O)C(C)C)cc1 ; "," ; *C1CC(=O)N(CCCCCC(=O)N[C@H](C(=O)N[C@H](*)CCCNC(N)=O)C(C)C)C1=O ; *CCCCCN1C(=O)CC(*)C1=O]

wherein the wavy lines indicate the points of attachment to the moiety that binds to SSTR2 and to the sarcophagine.

In another embodiment, the linker is of the following formula (IV):

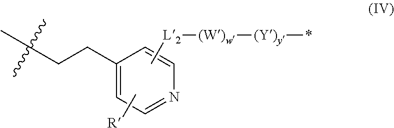

wherein
L′₂ is cycloalkylene-carbonyl, (C₂-C₆)alkylene, or (C₂-C₆)alkylene-carbonyl;
W′ is an amino acid unit;
w′ is an integer of 0 to 5;
Y′ is PAB-carbonyl with PAB being

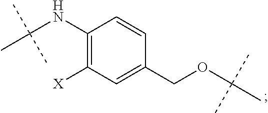

x can be H or

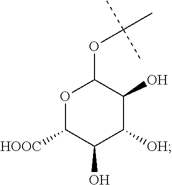

y′ is 0 or 1;
R′ is C₁-C₃ alkenyl or H.

In an embodiment the compound of formula (IV) is a compound of formula (IV′):

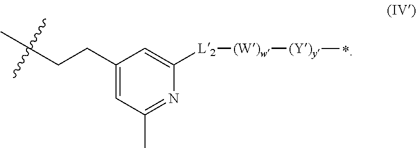

In an embodiment the compound of formula (IV′) is characterised with L₂′ being C₂ alkylene carbonyl and w′ being 2.

In an embodiment the linker of formula (IV′) is:

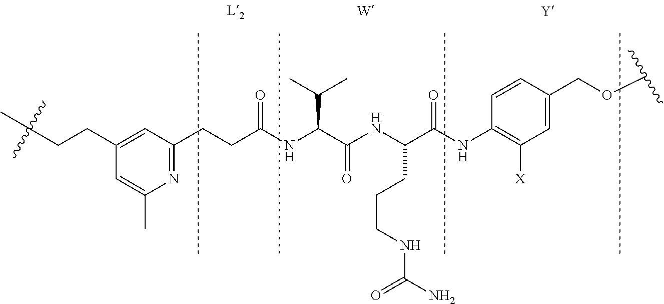

In another embodiment, the linker of formula (IV′) is:

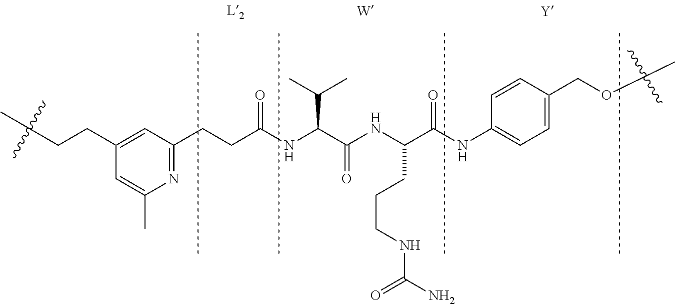 [SMILES: *CCc1cc(C)nc(CCC(=O)N[C@H](C(=O)N[C@@H](CCCNC(N)=O)C(=O)Nc2ccc(CO*)cc2)C(C)C)c1]

In another embodiment, the linker of formula (IV′) is:

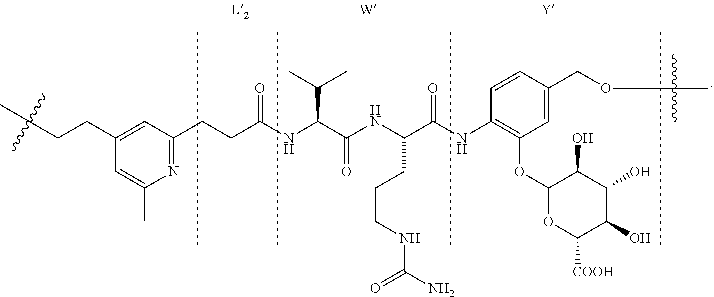

In a preferred embodiment, the linker “-L-” is unsubstituted propylene. Accordingly, in a further aspect the present invention provides a compound of Formula (IIa):

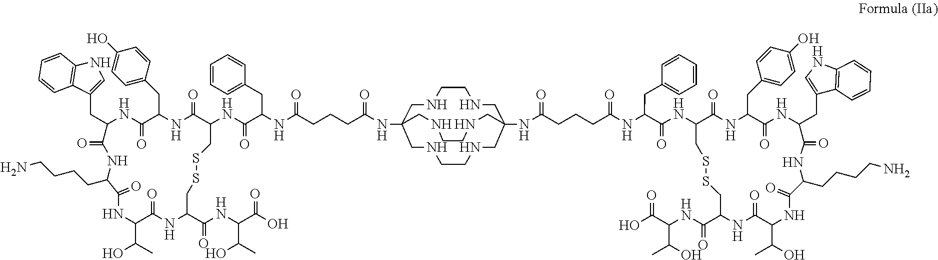 [SMILES: CC(O)C(NC(=O)C1CSSCC(NC(=O)C(Cc2ccccc2)NC(=O)CCCC(=O)NC23CNCCNCC(NC(=O)CCCC(=O)NC(Cc4ccccc4)C(=O)NC4CSSCC(C(=O)NC(C(=O)O)C(C)O)NC(=O)C(C(C)O)NC(=O)C(CCCCN)NC(=O)C(Cc5c[nH]c6ccccc56)NC(=O)C(Cc5ccc(O)cc5)NC4=O)(CNCCNC2)CNCCNC3)C(=O)NC(Cc2ccc(O)cc2)C(=O)NC(Cc2c[nH]c3ccccc23)C(=O)NC(CCCCN)C(=O)NC(C(C)O)C(=O)N1)C(=O)O]

or a pharmaceutically acceptable salt, complex, isomer, solvate or prodrug thereof.

In one embodiment, the compound of Formula (IIa) has the stereochemistry as depicted for the compound of Formula (IIb):

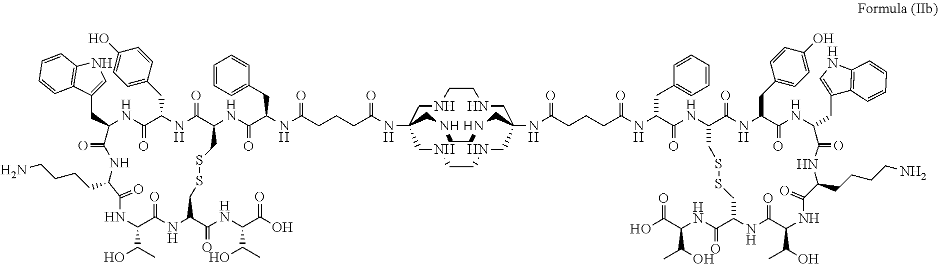

The present linkers may be synthesised using amide bond coupling. Many methods exist for amide synthesis. Some methods, but not limited to, are described in Montalbetti, Christian A. G. N (Tetrahedron 61 (46), 2005, 10827-10852). Alternatively, the linkers may be synthesized using standard stepwise addition of one or more residues using, for example, a peptide or protein synthesizer. Alternatively, other methods that may be used for amide formation includes, but not limited to, Beckmann rearrangement, Schmidt reaction, Nitrile hydrolysis, Willgerodt-Kindler reaction, Passerini reaction, Ugi reaction, Bodroux reaction, Chapman rearrangement, Leuckart amide synthesis, Ritter reaction, Ester aminolysis, Schotten-Baumann reaction, ruthenium based catalysis of alcohol and amine, or Photolytic addition of formamide to olefins.

“Alkyl” refers to a saturated monovalent hydrocarbon radical which may be straight chained or branched and preferably have from 1 to 10 carbon atoms or more preferably 1 to 6 carbon atoms or 1 to 9 carbon atoms (i.e., C₁-C₄ alkyl). Examples of such alkyl groups include methyl, ethyl, n-propyl, iso-propyl, n-butyl, iso-butyl, n-hexyl, and the like

“Alkylene” refers to divalent alkyl groups preferably having from 1 to 10 carbon atoms and more preferably 1 to 6 carbon atoms. Examples of such alkylene groups include methylene (—CH₂—), ethylene (—CH₂CH₂—), and the propylene isomers (e.g., —CH₂CH₂CH₂— and —CH(CH₃)CH₂—), and the like.

“Alkenylene” refers to a divalent hydrocarbon radical with at least one site of unsaturation, i.e., a carbon-carbon, sp² double bond, which may be straight chained or branched and preferably have from 2 to 10 carbon atoms and more preferably 2 to 6 carbon atoms and have at least 1 carbon to carbon double bond. An alkenylene radical includes radicals having “cis” and “trans” orientations, or alternatively, “E” and “Z” orientations. Examples include ethenyl (—CH═CH—), n-propenyl (—CH₂CH═CH—), iso-propenyl (—C(CH₃)═CH—), but-2-enyl (—CH₂CH═CHCH₂—), and the like.

“Alkynylene” refers to a linear or branched divalent hydrocarbon radical with at least one site of unsaturation, i.e., a carbon-carbon sp triple bond, preferably having from 2 to 10 carbon atoms and more preferably 2 to 6 carbon atoms and having at least 1 carbon to carbon triple bond. Examples of alkynyl groups include ethynyl (—C≡C—), propargyl (—CH₂C≡C—), pent-2-ynyl (—CH₂C≡CCH₂—CH₂—), and the like.

In this specification “optionally substituted” is taken to mean that a group may or may not be further substituted with one or more groups selected from hydroxyl, acyl, alkyl, alkoxy, alkenyl, alkenyloxy, alkynyl, alkynyloxy, amino, aminoacyl, thio, arylalkyl, arylalkoxy, aryl, aryloxy, carboxyl, acylamino, cyano, halogen, nitro, phosphono, sulfo, phosphorylamino, phosphinyl, heteroaryl, heteroaryloxy, heterocyclyl, heterocyclyloxy, oxyacyl, oxime, oxime ether, hydrazone, oxyacylamino, oxysulfonylamino, aminoacyloxy, trihalomethyl, trialkylsilyl, pentafluoroethyl, trifluoromethoxy, difluoromethoxy, trifluoromethanethio, trifluoroethenyl, mono- and di-alkylamino, mono- and di-(substituted alkyl)amino, mono- and di-arylamino, mono- and di-heteroarylamino, mono- and di-heterocyclyl amino, and unsymmetric di-substituted amines having different substituents selected from alkyl, aryl, heteroaryl and heterocyclyl, and the like, and may also include a bond to a solid support material, (for example, substituted onto a polymer resin). For instance, an “optionally substituted amino” group may include amino acid and peptide residues.

In an embodiment the “optionally substituted” group is selected from halo (e.g., chloro, fluoro or bromo), —CN, —NO₂, —CO₂H, —CO₂C₁₋₆alkyl, —CONH₂, —CONH(C₁₋₆alkyl), —CONH(C₁₋₆alkyl)₂, —OH, hydroxyC₁₋₆alkyl, C₁₋₆alkoxy, C₁₋₆alkyl, C₁₋₆acyl, carboxyC₁₋₆alkyl, acetyl, trifluoromethyl, benzyloxy, phenyl, phenoxy, —NH₂, —NH(C₁₋₆alkyl) or —N(C₁₋₆alkyl)₂.

As used herein, the term “residue” refers to a part of a compound resulting from the removal of one or more atoms. The one or more atoms to be removed may be hydrogen atoms. A person skilled in the art would understand, for example, where a compound comprises a carboxylic acid (—COOH) functional group, the residue that is found in the compound of Formula (I) comprises the carboxylate of the amino acid (i.e. —COO⁻), which is attached to the remainder of the compound.

As used herein, the term “pharmaceutically acceptable salts” refers to salts that retain the desired biological activity of the above-identified compounds, and include pharmaceutically acceptable acid addition salts and base addition salts. Suitable pharmaceutically acceptable acid addition salts of compounds of Formula (I) may be prepared from an inorganic acid or from an organic acid. Examples of such inorganic acids are hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic and arylsulfonic acids. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts may be prepared by reaction of a compound with the appropriate inorganic or organic acid via any of a number of known methods. Alternatively, alkali and alkaline earth metal salts can be prepared by reacting a compound with the appropriate base via a variety of known methods. The following are further examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates. Additional information on pharmaceutically acceptable salts can be found in Remington's Pharmaceutical Sciences, 19th Edition, Mack Publishing Co., Easton, PA 1995. In the case of agents that are solids, it is understood by those skilled in the art that the inventive compounds, agents and salts may exist in different crystalline or polymorphic forms, all of which are intended to be within the scope of the present invention and specified formulae.

As used herein, the term “complex” refers to a moiety comprising a ligand and a metal coordinated with a suitable part of the ligand. For example, the compounds of Formula (I) as disclosed herein acts as a ligand for one or more metal ions, where the metal ion is coordinated to the ligand via the metal chelator.

As used herein, used herein, the term “isomer” refers to and includes all regioisomers and stereoisomers of the compounds of the present invention. Examples of stereoisomers include diastereomers and enantiomers, where appropriate.

Examples of ions that may form complexes with the compounds of the present invention includes ions of a metal selected from the group consisting of Cu, Tc, Gd, Ga, In, Co, Re, Fe, Mg, Ag, Rh, Pt, Cr, Ni, V, Ir, Zn, Cd, Mn, Ru, Pd, Hg, Ti, Lu, Sc, Zr, Lu, Sc, Zr, Y, Ac, As, Ra and Pb.

In some embodiments, the metal ion complexed in the metal chelator is a radionuclide.

In some embodiments, the compound of Formulae (I) or (II) is complexed with a radionuclide selected from the group consisting of ⁶⁰Cu, ⁶¹Cu, ⁶²Cu, ⁶⁴Cu, ⁶⁷Cu, ⁶⁸Ga, ⁹⁰Y, ¹¹¹In, ¹⁷⁷Lu, ¹⁸⁸Re, ²¹¹As, ²¹²Pb and ²²⁵Ac.

As used herein, the term “solvate” refers to a complex of the compound, where the complex may have variable stoichiometry formed by a solute and a solvent. Such solvents in the solvate should not interfere with the biological activity of the solute. Examples of suitable solvents may include water, ethanol or acetic acid. Methods of solvation of the compound are generally known in the art.

As used herein, the term “prodrug” refers to and includes derivatives that are converted in vivo to the compounds of the present invention. Such derivatives would readily occur to those skilled in the art, and include, for example, compounds containing a free hydroxyl group that is converted into an ester derivative, or containing a ring nitrogen atom that is converted to an N-oxide. Examples of ester derivatives include alkyl esters, phosphate esters and those formed from amino acids.

As used herein the terms “treating”, “treatment”, “preventing”, “prevention” and grammatical equivalents refer to any and all uses which remedy the stated neuroendocrine tumour, prevent, retard or delay the establishment of the disease, or otherwise prevent, hinder, retard, or reverse the progression of the disease. Thus the terms “treating” and “preventing” and the like are to be considered in their broadest context. For example, treatment does not necessarily imply that a patient is treated until total recovery. Where the disease displays or a characterized by multiple symptoms, the treatment or prevention need not necessarily remedy, prevent, hinder, retard, or reverse all of said symptoms, but may prevent, hinder, retard, or reverse one or more of said symptoms.

As used herein, the term “cancer” broadly encompasses neoplastic diseases characterised by abnormal cell growth with the potential to invade or spread to other parts of the body. The cancer may be benign, which does not spread to other parts of the body. The cancer may be malignant, meaning that the cancer cells can spread through the circulatory system or lymphatic system. The term as used herein includes all malignant, i.e. cancerous, disease states. The cancer may be present as a tumour. Accordingly, the term “tumour” is used generally to define any malignant cancerous or pre-cancerous cell growth, and may include leukemias, but is particularly directed to solid tumours or carcinomas.

In certain embodiments, the cancer is associated with expression of somatostatin type 2 receptors (SSTR2). In specific embodiments, the cancer is selected from the group consisting of pituitary tumours, neuroendocrine tumours, renal cell cancer, breast cancer, meningioma, glioma, neuroblastoma, colorectal cancer, pheochromocytoma, medullary thyroid cancer, ovarian cancer, head and/or neck cancer, gastric cancer, adrenal cancer, brain cancer, and a hematologic malignancy such as lymphoma or leukaemia. In particular embodiments, the cancer is a neuroendocrine tumour, such as a carcinoid tumour in the lung, appendix, digestive tract, prostate, thymus or rectum or a pancreatic neuroendocrine tumour. In further embodiments, the cancer is a neuroendocrine tumour such as a gastrinoma, insulinoma or non-functioning islet cell tumour.

In some embodiments, the cancer is selected from the group consisting of epithelial ovarian cancer, ovarian carcinoma, osteosarcoma, pancreatic adenocarcinoma, colorectal cancer, lung cancer, non-small cell lung cancer, gastric cancer, endometrial carcinoma, pancreatic adenocarcinoma, medullary thyroid carcinoma, differentiated thyroid cancer, breast cancer, invasive ductal carcinoma of the breast, oral squamous cell carcinoma, esophageal cancer, renal cell cancer, insulinoma, prostate cancer, neuroendocrine differentiated prostate cancer, pheochromocytoma, adenoid cystic cancer, hepatocellular carcinoma, cervical cancer, small intestine cancer, neuroendocrine tumour, anal cancer, chordoma, desmoid tumour, head and neck cancer, thymus cancer, pancreatic cancer, cholangiocellular carcinoma, esophageal cancer, salivary gland cancer, sarcoma and carcinoma of unknown primary cancer.

As used herein, the term “expression” in relation to SSTR2 relates to the presentation of the receptor on the surface of the tumor or cancer cell. Healthy tissue may also express SSTR2, whereas cancerous or tumor cells and/or tissues may show upregulation or “overexpression” of SSTR2 meaning that the abundance of the receptor on cancerous cells is greater than when compared to healthy tissue.

The present invention contemplates the use of the compounds of Formulae (I) or (II) complexed with a suitable radionuclide for the treatment of a cancer in a subject. In some embodiments, a compound of Formulae (I) or (II) complexed with a radionuclide may be used for the treatment of a cancer that is associated with expression of SSTR2. In other embodiments, a compound of the present invention complexed with a radionuclide is used for the treatment of a cancer selected from the group consisting of pituitary tumours, neuroendocrine tumours, renal cell cancer, breast cancer, meningioma, glioma, neuroblastoma, colorectal cancer, pheochromocytoma, medullary thyroid cancer, ovarian cancer, head and/or neck cancer, gastric cancer, adrenal cancer, brain cancer, and a hematologic malignancy such as lymphoma or leukaemia. In particular embodiments, the cancer is a neuroendocrine tumour, such as a carcinoid tumour in the lung, appendix, digestive tract, prostate, thymus or rectum or a pancreatic neuroendocrine tumour. In further embodiments, the cancer is a neuroendocrine tumour such as a gastrinoma, insulinoma or non-functioning islet cell tumour.

The present invention also discloses the use of the compounds of Formulae (I) or (II) complexed with a suitable radionuclide for radioimaging a subject. In certain embodiments, the compounds of Formulae (I) or (II) complexed with a suitable radionuclide is used for radioimaging a cancer that is associated with expression of SSTR2. In other embodiments, a compound of the present invention is used for the radioimaging of a cancer selected from the group consisting of pituitary tumours, neuroendocrine tumours, renal cell cancer, breast cancer, meningioma, glioma, neuroblastoma, colorectal cancer, pheochromocytoma, medullary thyroid cancer, ovarian cancer, head and/or neck cancer, gastric cancer, adrenal cancer, brain cancer, and a hematologic malignancy such as lymphoma or leukaemia. In particular embodiments, the cancer is a neuroendocrine tumour, such as a carcinoid tumour in the lung, appendix, digestive tract, prostate, thymus or rectum or a pancreatic neuroendocrine tumour. In further embodiments, the cancer is a neuroendocrine tumour such as a gastrinoma, insulinoma or non-functioning islet cell tumour.

The term “subject” as used herein refers to mammals and includes humans, primates, livestock animals (e.g. sheep, pigs, cattle, horses, donkeys), laboratory test animals (e.g. mice, rabbits, rats, guinea pigs), performance and show animals (e.g. horses, livestock, dogs, cats), companion animals (e.g. dogs, cats) and captive wild animals. Preferably, the mammal is human or a laboratory test animal. Even more preferably, the mammal is a human.

The term “therapeutically effective amount” or “effective amount” is an amount sufficient to effect beneficial or desired clinical results. An effective amount can be administered in one or more administrations. For the purposes of radioimaging, an effective amount is sufficient for an image showing the localisation of the compound of Formulae (I) or (II) administered to the subject, owing to the detection of the products of decay from the radioisotope that is complexed with the compound. For the purposes of treatment, an effective amount is typically sufficient to palliate, ameliorate, stabilize, reverse, slow and/or delay the progression of the cancer.

The compounds of the present invention complexed with a radionuclide may be administered to a subject in need thereof as a composition by a parenteral route. Administration by intravenous injection may be preferred. Alternatively, the formulations of the present invention may be given by intra-arterial or other routes, for example, via intrathecal, intratumoural, or intra-peritoneal routes. It will be appreciated that the route of administration may vary depending on the type of cancer being diagnosed or treated. In one embodiment, the cancer being diagnosed or treated is brain cancer and the compound of Formula (I) complexed with a radionuclide is administered intratumorally or intrathecally. In certain embodiments, the compounds of the present invention complexed with a radionuclide may be administered intravenously and the compound is allowed to circulate and localize at the cancer site. In other embodiments, the compounds of the present invention complexed with a radionuclide may be administered directly to a cancer site in a subject or administered to a blood vessel that leads directly to the cancer site. The present inventors believe that the administration of a compound complexed with a radionuclide directly to a cancer may result in an improved therapeutic effect the administered dose is not necessarily subjected to elimination processes within the subject. This in turn may result in a smaller dose of the compound being required for a therapeutic effect, which is a more cost-effective approach to cancer treatment.

The methods for the treatment of a cancer as disclosed herein may also include the further administration of one more therapeutically effective agents. Other therapeutically effective agents include chemotherapy agents, other radiotherapeutic and/or radioimaging agents, immune checkpoint inhibitors, nucleic acid therapeutics, cancer vaccines, RNAi therapeutics, siRNA therapeutics and mRNA therapeutics.

In certain embodiments, the method further comprises the administration of one or more therapeutically effective agents. In some embodiments, the therapeutically effective agent is a chemotherapy agent. In some embodiments, the therapeutically effective agent is a radiotherapeutic agent. In other embodiments, the therapeutically effective agent is a radioimaging agent. In another embodiment, the therapeutically effective agent is an immune checkpoint inhibitor.

In some embodiments, the immune checkpoint inhibitor is an antibody or fragment thereof. In some embodiments, the immune checkpoint inhibitor is associated with PD-1, PD-L1 or CTLA-4. In other embodiments, the immune checkpoint inhibitor is associated with PD-L2, B7-H3, B7-H4, BTLA, IDO, KIR, LAG3, A2AR, TIM-3 or VISTA. In certain embodiments, the one or more immune checkpoint inhibitors is an antibody against an immune checkpoint protein. In some embodiments, the one or more immune checkpoint inhibitors is an anti-PD-L1 antibody, anti-PD-1 antibody or anti-CTLA-4 antibody. In some embodiments the one or more immune checkpoint inhibitors is selected from the group consisting of durvalumab, avelumab, ipilimumab, nivolumab, pembrolizumab, atezolizumab, cemiplimab, envafolimab, BMS-936559, CK-301, CS-1001, and SHR-1316.

As used herein, the term “immune checkpoint inhibitor” refers to a compound that modulates the function of the immune system of a subject. Cancer cells are able to proliferate since they have the ability to bypass a patient's immune system at various “checkpoints” by binding to and deactivating immune cells that would otherwise target and destroy cancer cells. In the context of cancer therapy, immune checkpoint inhibitors and their administration in order to inhibit the immunosuppressive effect that a cancer cell may have on the immune system of a patient are well known. Immune checkpoint inhibitors are often proteins that bind to either the tumour cell or a cell of the immune system in order to prevent the deactivation of the patient's immune system. Conversely, an immune checkpoint inhibitor may be considered to enhance the immune response generated in the presence of an antigen, i.e., a cancer cell. Immune checkpoint proteins include PD-1 (also known as CD279), PD-L1 (also known as CD274), CTLA-4, A2AR, B7-H3 (also known as CD276), B7-H4 (also known as VTCN1), BTLA (also known as CD272), IDO, KIR, LAG3, TIM-3 and VISTA. Immune checkpoint inhibitors may be specific for a particular immune checkpoint protein, i.e., anti-immune checkpoint antibodies. Such antibodies include anti-CTLA4 antibodies (e.g., ipilimumab, tremelimumab), anti-PD-1 antibodies (e.g., nivolumab, pembrolizumab, pidilizumab, tislelizumab, and RG7446), and anti-PD-L1 antibodies (e.g., BMS-93655, MPDL3280A, MSBOO10718C and MED14736). Preferred immune checkpoint inhibitors are antibodies that bind to a specific immune checkpoint protein, whether the protein is the immune checkpoint protein itself, a receptor thereof or a ligand thereof.

In some embodiments, the one or more immune checkpoint inhibitor is an antibody or fragment thereof. Suitable antibodies include an anti-PD1 antibody or fragment thereof, an anti-PDL1 antibody or fragment thereof and an anti-CTLA4 antibody or fragment thereof. Accordingly, in some embodiments, the checkpoint inhibitor is associated with PD-1, PD-L1 or CTLA-4.

In other embodiments, the checkpoint inhibitor is associated with PD-L2, B7-H3, B7-H4, BTLA, IDO, KIR, LAG3, A2AR, TIM-3 or VISTA.

In certain embodiments, the one or more immune checkpoint inhibitor is an antibody against an immune checkpoint protein.

In some embodiments, the one or more immune checkpoint inhibitor is selected from the group consisting of durvalumab, avelumab, ipilimumab, nivolumab, pembrolizumab, atezolizumab, cemiplimab, envafolimab, BMS-936559, CK-301, CS-1001, and SHR-1316.

| Immune checkpoint inhibitor | Target |
|---|---|
| atezolizumab | PD-L1 |
| durvalumab | PD-L1 |
| avelumab | PD-L1 |
| envafolimab | PD-L1 |
| BMS-936559 | PD-L1 |
| CK-301 | PD-L1 |
| CS-1001 | PD-L1 |
| SHR-1316 (adebrelimab) | PD-L1 |
| nivolumab | PD-1 |
| pembrolizumab | PD-1 |
| cemiplimab | PD-1 |
| ipilimumab | CTLA4 |

The compounds are typically used in the form of pharmaceutical compositions that are formulated depending on the desired mode of administration. The compositions are prepared in manners well known in the art.

In the above embodiments, the compositions of the present invention comprise ethanol as a component. The ethanol used in the composition may be anhydrous ethanol. Alternatively, the ethanol used in the composition may not have been subject to drying processes and may be hydrated. The ethanol is preferably pharmaceutical grade ethanol. The ethanol present in the composition may assist in preventing radiolysis of the radiolabelled complex of Formula (I).

In the above embodiments, the compositions of the present invention also comprise sodium chloride as a component. The sodium chloride in the formulations of the present invention may be provided as a saline solution. A saline solution is defined as an aqueous solution of sodium chloride. For example, normal saline is defined as an aqueous solution of sodium chloride at a concentration of 0.9% (w/v). In an embodiment of the present invention, the sodium chloride of a formulation is provided by a saline solution.

In the above embodiments, the compositions of the present invention comprise gentisic acid, or pharmaceutically acceptable salts and/or hydrates thereof, as a component. Gentisic acid is also known as 2,5-dihydroxybenzoic acid, 5-hydroxysalicylic acid or hydroquinonecarboxylic acid. Salts of gentisic acid may include the sodium salt and the sodium salt hydrate. Any reference to gentisic acid may include a reference to salts thereof, where relevant. It has been identified by the present inventors that the gentisic acid or salt thereof, within the present composition may assist in preventing or minimising radiolysis of the radiolabelled complex of Formula (I).

The invention in other embodiments provides a pharmaceutical pack or kit comprising one or more containers filled with one or more of the ingredients of the pharmaceutical compositions of the invention. In such a pack or kit can be found at least one container having a unit dosage of the agent(s). Conveniently, in the kits, single dosages can be provided in sterile vials so that the clinician can employ the vials directly, where the vials will have the desired amount and concentration of compound and radionuclide, which may be admixed prior to use. Associated with such container(s) can be various written materials such as instructions for use, or a notice in the form prescribed by a governmental agency regulating the manufacture, use or sale of pharmaceuticals, imaging agents or biological products, which notice reflects approval by the agency of manufacture, use or sale for human administration.

The compounds of the invention may be used or administered in combination with one or more additional drug(s) that are anti-cancer drugs and/or procedures (e.g. surgery, radiotherapy) for the treatment of the disorder/diseases mentioned. The components can be administered in the same formulation or in separate formulations. If administered in separate formulations the compounds of the invention may be administered sequentially or simultaneously with the other drug(s).

In addition to being able to be administered in combination with one or more additional drugs that include anti-cancer drugs, the compounds of the invention may be used in a combination therapy. When this is done, the compounds are typically administered in combination with each other. Thus one or more of the compounds of the invention may be administered either simultaneously (as a combined preparation) or sequentially in order to achieve a desired effect. This is especially desirable where the therapeutic profile of each compound is different such that the combined effect of the two drugs provides an improved therapeutic result.

Pharmaceutical compositions of this invention for parenteral injection comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminium monostearate and gelatin.

If desired, and for more effective distribution, the compounds can be incorporated into slow release or targeted delivery systems such as polymer matrices, liposomes, and microspheres.

The injectable formulations can be sterilized, for example, by filtration through a bacterial-retaining filter, or by incorporating sterilizing agents in the form of sterile solid compositions that can be dissolved or dispersed in sterile water or other sterile injectable medium just prior to use.

In another aspect, the present invention also provides a process for the preparation of compounds of Formula (I) or a pharmaceutically acceptable salt thereof as defined herein.

The compounds of Formula (I) may be prepared by a series of peptide coupling steps using appropriate amine and carboxylic acid derivatives. The coupling partners may require the installation of one or more protecting groups, which are subsequently removed after the coupling reaction. A list of suitable protecting groups in organic synthesis and procedures for their installation and removal can be found in T. W. Greene's Protective Groups in Organic Synthesis, 3rd Edition, John Wiley & Sons, 1991. Where a peptide coupling step is used to prepare the compounds of the present invention or a precursor thereof, the reaction may be performed under solution phase or solid phase conditions in the presence of one or more bases or other reagents as required. The coupling partners used in a given reaction step may be modified with one or more suitable groups that will facilitate the coupling reaction, for example, a leaving group. The preparation of compounds of Formula (I) may include the selection and installation of one or more protecting groups or the like that facilitate the coupling of components with the required site selectivity.

For example, the steps required to prepare a compound of Formula (I) may include the installation of one or more nitrogen or oxygen protecting groups installed at one or more amine or carboxylic acid functional groups.

As used herein, the term “oxygen protecting group” refers to a group that can prevent the oxygen moiety reacting during further derivatisation of the protected compound and which can be readily removed when desired. In one embodiment, the protecting group is removable in the physiological state by natural metabolic processes. Examples of oxygen protecting groups include acyl groups (such as acetyl), ethers (such as methoxy methyl ether (MOM), α-methoxy ethoxy methyl ether (MEM), p-methoxy benzyl ether (PMB), methylthio methyl ether, pivaloyl (Piv), tetrahydropyran (THP)), and silyl ethers (such as trimethylsilyl (TMS) tert-butyl dimethyl silyl (TBDMS) and triisopropylsilyl (TIPS) groups.

As used herein, the term “nitrogen protecting group” refers to a group that can prevent the nitrogen moiety reacting during further derivatisation of the protected compound and which can be readily removed when desired. In one embodiment, the protecting group is removable in the physiological state by natural metabolic processes and in essence the protected compound is acting as a prodrug for the active unprotected species. Examples of suitable nitrogen protecting groups that may be used include formyl, trityl, phthalimido, acetyl, trichloroacetyl, chloroacetyl, bromoacetyl, iodoacetyl; urethane-type blocking groups such as benzyloxycarbonyl (CBz), 4-phenylbenzyloxycarbonyl, 2-methylbenzyloxycarbonyl, 4-methoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxycarbonyl, t-butoxycarbonyl (tBoc), 2-(4-xenyl)-isopropoxycarbonyl, 1,1-diphenyleth-1-yloxycarbonyl, 1,1-diphenylprop-1-yloxycarbonyl, 2-phenylprop-2-yloxycarbonyl, 2-(p-toluyl)-prop-2-yloxy-carbonyl, cyclo-pentanyloxy-carbonyl, 1-methylcyclopentanyloxycarbonyl, cyclohexanyloxycarbonyl, 1-methylcyclohexanyloxycarbonyl, 2-methylcyclohexanyloxycarbonyl, 2-(4-toluylsulfono)-ethoxycarbonyl, 2-(methylsulfono) ethoxycarbonyl, 2-(triphenylphosphino)-ethoxycarbonyl, fluorenylmethoxycarbonyl (Fmoc), 2-(trimethylsilyl) ethoxycarbonyl, allyloxycarbonyl, 1-(trimethylsilylmethyl) prop-1-enyloxycarbonyl, 5-benzisoxalylmethoxy carbonyl, 4-acetoxybenzyloxycarbonyl, 2,2,2-trichloroethoxycarbonyl, 2-ethynyl-2-propoxycarbonyl, cyclopropylmethoxycarbonyl, 4-(decyloxy)benzyloxycarbonyl, isobornyloxycarbonyl, 1-piperidyloxycarbonlyl and the like; benzoylmethylsulfono group, 2-nitrophenylsulfenyl, diphenylphosphine oxide, and the like. The actual nitrogen protecting group employed is not critical so long as the derivatised nitrogen group is stable to the condition of subsequent reaction(s) and can be selectively removed as required without substantially disrupting the remainder of the molecule including any other nitrogen protecting group(s). Further examples of these groups are found in: Greene, T. W. and Wuts, P. G. M., Protective Groups in Organic Synthesis, Second edition; Wiley-Interscience: 1991; Chapter 7; McOmie, J. F. W. (ed.), Protective Groups in Organic Chemistry, Plenum Press, 1973; and Kocienski, P. J., Protecting Groups, Second Edition, Thieme Medical Pub., 2000.

The compounds of Formulae (I), (II), (IIa) or (IIb) or pharmaceutically acceptable salts thereof as disclosed herein may be synthesised by coupling the moiety that binds to SSTR2 to the desired linkers (where present) and subsequently, to the chelator. Various protection and deprotection steps may also be employed, where the conditions for each step are compatible with the remainder of the compound. An exemplary synthetic scheme is provided in Scheme 1:

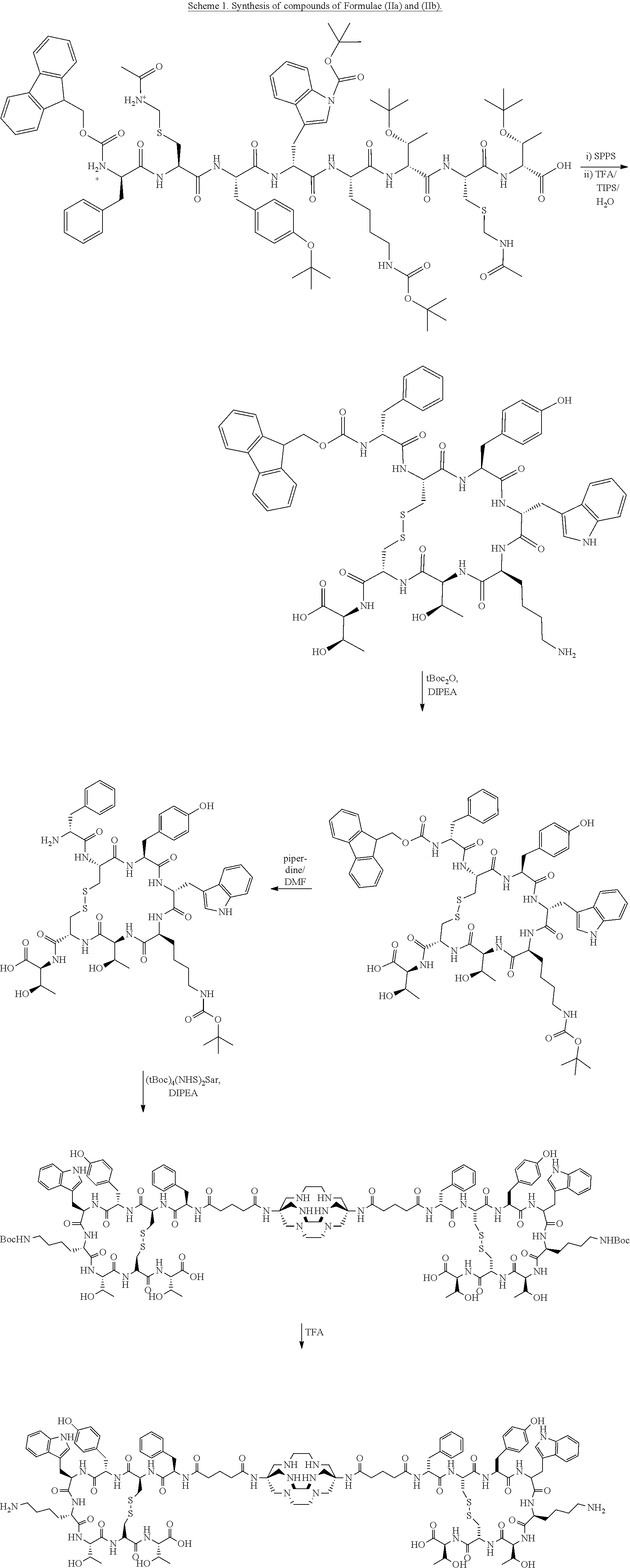

Scheme 1 describes a synthesis of a compound of Formula (II) where the sarcophagine is coupled with two moieties of the linker-octreotate group at the same time. The reaction may be performed under standard peptide coupling conditions with a suitable peptide coupling reagent and a base, where the linker-octreotate group contains the amine functional group and the metal chelator contains the carboxylic acid functional group that will participate in the coupling reaction.

Alternatively, the compounds of Formulae (I), (II) and (IIa), or pharmaceutically acceptable salts thereof, as disclosed herein may be synthesised according to Scheme 2:

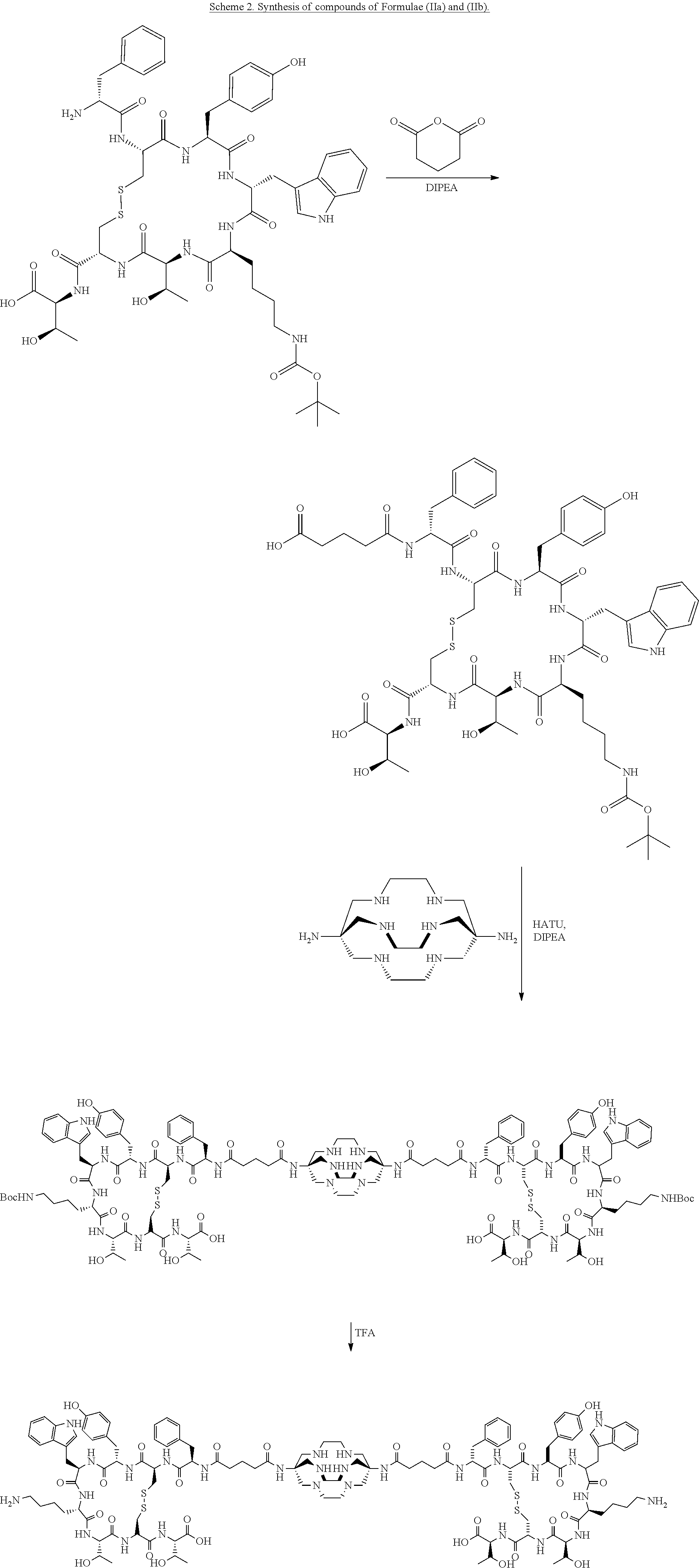

Scheme 2 also depicts a particular embodiment of a process as described herein. Two moieties of the linker-octreotate group are coupled with a sarcophagine, however in this synthetic route, the amine group that participates in the coupling reaction is found on sarcophagine, while the carboxylic acid group is found on the linker-octreotate group. The reaction may also be performed under standard peptide coupling reaction conditions with a peptide coupling reagent and a base.

In certain embodiments, the present invention provides a process for preparing a compound of Formula (I) or a pharmaceutically acceptable salt thereof as described above, wherein the process includes one or more peptide coupling steps. The process may also include one or more protection and deprotection steps where appropriate. In other embodiments, the process for preparing a compound of Formula (I) includes a peptide coupling step performed under solution phase conditions with one or more peptide coupling reagents and one or more bases.

Variation in the structure of the sarcophagine and subsequently the nature and the location of one or more functional groups will naturally dictate the synthetic route required to access the compounds of Formula (I). For example, where the terminal group on the sarcophagine is oxygen-based, e.g. a carboxylic acid, then the protecting group (if required) and subsequent reaction should be compatible and allow for coupling to provide compounds of Formula (I). In contrast, where the terminal group on the sarcophagine is nitrogen-based, e.g. an amine, then the protecting groups (where necessary) present on either of the coupling partners should be compatible with the subsequent synthetic steps to provide access to compounds of Formula (I).

The synthetic schemes depicted herein show the coupling of the linker-octreotate group to the two terminal amine groups of sarcophagine. The synthetic schemes disclosed herein encompass modifications of a protection/deprotection strategy that allow for the coupling of the linker-SSTR2 binding moiety fragments at the desired centres to provide compounds of Formula (I).

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

### EXAMPLES

The following examples are illustrative of the disclosure and should not be construed as limiting in any way the general nature of the disclosure of the description throughout this specification.

### Synthesis of Compounds of the Invention

Various starting materials and other reagents may be purchased from commercial suppliers, such as Aldrich Chemical Company or Lancaster Synthesis Ltd., and used without further purification, unless otherwise indicated. If needed, solvents may be purified by using standard methods in the art. [Co((NO₂)₂sar)]Cl₃, [Co((NH₂)₂sar)]Cl₃, (NH₂)₂sar, [Cu(NH₃)₂sar](CF₃SO₃)₄ may be prepared according to established procedures. (1) Geue, R. J.; Hambley, T. W.; Harrowfield, J. M.; Sargeson, A. M.; Snow, M. R. J. Am. Chem. Soc. 1984, 106, 5478-5488. (2) Bottomley, G. A.; Clark, I. J.; Creaser, I. I.; Engelhardt, L. M.; Geue, R. J.; Hagen, K. S.; Harrowfield, J. M.; Lawrance, G. A.; Lay, P. A.; Sargeson, A. M.; See, A. J.; Skelton, B. W.; White, A. H.; Wilner, F. R. Aust. J. Chem. 1994, 47, 143-179 and (3) Bernhardt, P. V.; Bramley, R.; Engelhardt, L. M.; Harrowfield, J. M.; Hockless, D. C. R.; Korybut-Daszkiewicz, B. R.; Krausz, E. R.; Morgan, T.; Sargeson, A. M.; Skelton, B. W.; White, A. H. Inorg. Chem. 1995, 34, 3589-3599.

The reactions set forth below are performed under a positive pressure of nitrogen, argon or with a drying tube, at ambient temperature (unless otherwise stated), in anhydrous solvents, and the reaction flasks are fitted with rubber septa for the introduction of substrates and reagents via syringe.

Work-ups are typically done by doubling the reaction volume with the reaction solvent or extraction solvent and then washing with the indicated aqueous solutions using 25% by volume of the extraction volume (unless otherwise indicated). Product solutions are dried over anhydrous sodium sulfate prior to filtration, and evaporation of the solvents was under reduced pressure on a rotary evaporator and noted as solvents removed in vacuo. Flash column chromatography [Still et al, J. Org. Chem., 43, 2923 (1978)] is conducted using E Merck-grade flash silica gel (47-61 mm) and a silica gel: crude material ratio of about 20:1 to 50:1, unless otherwise stated. Hydrogenolysis is done at the pressure indicated or at ambient pressure.

Mass spectra may be recorded in the positive ion mode on an Agilent 6510 Q-TOF LC/MS Mass Spectrometer coupled to an Agilent 1100 LC system (Agilent, Palo Alto, CA). Data is acquired and reference mass corrected via a dual-spray electrospray ionisation source, using the factory-defined calibration procedure. Each scan or data point on the Total Ion Chromatogram is an average of 9652 transients, producing 1.02 scans s⁻¹. Spectra are created by averaging the scans across each peak. Mass spectrometer conditions: fragmentor: 200-300 V; drying gas flow: 7 L/min; nebuliser: 30 psi; drying gas temp: 325° C.; Vcap: 4000 V; skimmer: 65 V; OCT R_{f}V: 750 V; scan range acquired: 150-3000 m/z.

HPLC-MS traces may be recorded using an Agilent Eclipse Plus C18 column (5 μm, 2.1×150 mm) coupled to the Agilent 6510 Q-TOF LC/MS Mass Spectrometer described above. 1 μL aliquots of each sample were injected onto the column using the Agilent 1100 LC system, with a flow rate of 0.5 mL/min. Data acquisition parameters are the same as those described above for mass spectra, with the exception of the fragmentor (fragmentor voltage: 100 V).

NMR spectra may be recorded on a Varian FT-NMR 500 spectrometer operating at 500 MHz for ¹H NMR and 125.7 MHz for ¹³C-NMR. NMR spectra are obtained as D₂O solutions (reported in ppm), using acetone as the reference standard (2.22 ppm and 30.89 ppm respectively). Other NMR solvents can be used as needed. When peak multiplicities are reported, the following abbreviations are used: s=singlet, d=doublet, t=triplet, m=multiplet, br=broadened, dd=doublet of doublets, dt=doublet of triplets. Coupling constants, when given, are reported in Hertz.

Semi-preparative HPLC purifications may be performed using an Agilent 1200 Series HPLC system with a 5 mL/min flow rate. Solvent gradients and column specifications are described in the examples. An automated Agilent 1200 fraction collector collected 1-3 mL fractions and fraction collection was based on UV-Vis detection at 214 or 220 nm, with a lower threshold limit between 100-400 mAU. Each fraction was analysed using MS and analytical HPLC.

### Example 1: Preparation of SARbisTATE

### Tyr³-Octreotate

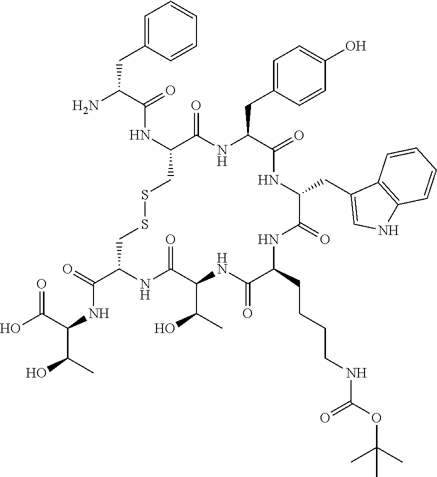 [SMILES: C[C@@H](O)[C@H](NC(=O)[C@@H]1CSSC[C@H](NC(=O)[C@H](N)Cc2ccccc2)C(=O)N[C@@H](Cc2ccc(O)cc2)C(=O)N[C@H](Cc2c[nH]c3ccccc23)C(=O)N[C@@H](CCCCNC(=O)OC(C)(C)C)C(=O)N[C@@H]([C@@H](C)O)C(=O)N1)C(=O)O]

The linear [Tyr³]-octreotate peptide (dPhe-Cys-Tyr-dTrp-Lys-Thr-Cys-Thr-OH is synthesised on 2-chlorotrityl chloride resin using standard Fmoc solid phase peptide synthesis procedures. An excess of resin (0.06 g, ˜0.8 mmol/g) was swelled in N,N-dimethylformamide (DMF), followed by sequential coupling of the amino acid residues. Fmoc-deprotection is performed by treating the peptide-resin with 20% piperidine/DMF (v/v; 5 ml). The reaction supernatant is then drained and the resin washed with DMF (3×5 mL) and dichloromethane (DCM) (3×5 mL). The resin is transferred to a falcon tube and trifluoroacetic acid (5 mL), deionised water (0.15 mL) and triisopropylsilane (0.15 mL) are added. The falcon tube is placed on a shaker for 40 min. The peptide material is precipitated from the solution using diethyl ether (15 mL) and the mixture is centrifuged (3 mins, 3000 rpm). The supernatant is discarded and the precipitate dissolved in A: B (70:30). This solution is filtered (MilliQ 0.45 μm syringe filter) and lyophilised.

The crude peptide material is purified by semi-preparative reverse phase HPLC (Eclipse XDB-C18 5 μm 9.5×250 mm column) using a linear 1% A→B/min gradient. The linear peptide is determined by ESI-MS and fractions containing the linear peptide are lyophilised. The dried fractions are then redissolved in ammonium acetate (25 mM, pH 6.5, 8 mL) and an excess of 2,2-dithiodipyridine (12 mg) is added. The solution is then applied to a semi-preparative reverse phase HPLC column, and purified using a linear 1% A→B/min gradient. Cyclic peptide is identified and fractions containing the cyclic peptide are lyophilised.

The cyclic peptide is then reacted with (tBoc)₄(NHS)₂Sar in the presence of diisopropylethylamine to produce the Boc-protected SAR-bisTATE compound. Global deprotection of the compound to remove the Boc groups was performed in the presence of TFA.

### Example 2: Biodistribution of ⁶⁴Cu-SARbisTATE in SSTR2-Positive Tumour Bearing Mice

Fifty eight female mice (BALB/c-Foxn1ν mice, age 8 to 12 weeks) were inoculate subcutaneously on the right flank with 3 million AR42J (rat SSTR2 expressing pancreatic cancer cell line) cells in PBS: Matrigel (1:1). Mice were weighed and tumours measured twice weekly using electronic callipers. Tumour volume (mm³) was calculated as length×width×height×π/6. Mice were randomised into tumour-volume matched groups of the biodistribution study (tumour volume range: 54-411 mm³, group means 265 mm³).

SARbisTATE was labelled with ⁶⁴Cu and injected into mice via tail vein injection in saline. Parallel groups (n=4-6) were harvested for tissue biodistribution and blood via cardiac puncture at 1 h, 4, h, 24 h and 48 h post-injection. Biodistribution tissues were excised, weighed and counted using a Capintec (Captus 4000e) gamma counter. The data were analysed using Prism 9 for Windows (GraphPad).

1. A compound of Formula (I): 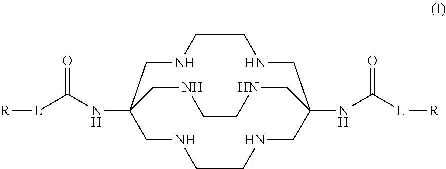
or a pharmaceutically acceptable salt, complex, isomer, solvate or prodrug thereof,
wherein
each R is a moiety that binds to SSTR2; and
each -L- is a linker moiety conjugated to the moieties that binds to SSTR2 and to the sarcophagine.

2. The compound according to claim 1, or a pharmaceutically acceptable salt, complex, isomer, solvate or prodrug thereof, wherein each R is independently selected from the group consisting of octreotide, lanreotide, pasireotide, octreotate and combinations thereof.

3. A compound of Formula (II): 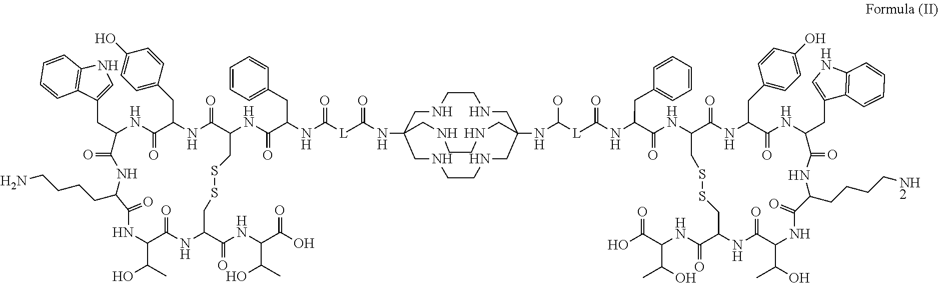
or a pharmaceutically acceptable salt, complex, isomer, solvate or prodrug thereof,
wherein -L- is a linker moiety.

4. A compound according to any one of claims 1 to 3, or a pharmaceutically acceptable salt, complex, isomer, solvate or prodrug thereof, wherein each -L- is independently an optionally substituted —C₁-C₁₀alkylene-, —C₂-C₁₀alkenylene- or —C₂-C₁₀alkynylene-group, one or more amino acids residues, one or more PEG groups, or combinations thereof; wherein one or more of the carbon atoms in the alkylene, alkenylene or alkynylene group may be replaced with NH, S, O, a C₅-C₈ aromatic or aliphatic cyclic group or a C₅-C₈ aromatic or aliphatic heterocyclic group.

5. A compound according to claim 3, wherein the compound of Formula (II) has the structure of Formula (IIa): 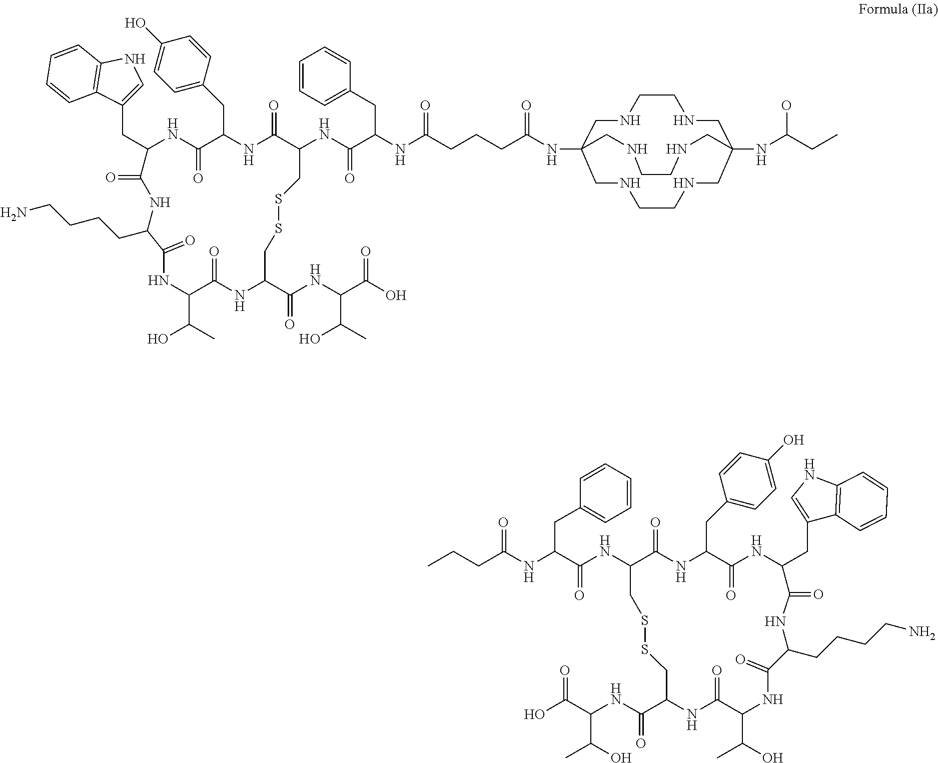
or a pharmaceutically acceptable salt, complex, isomer, solvate or prodrug thereof.

6. A compound according to claim 3, wherein the compound of Formula (II) has the structure of Formula (IIb): 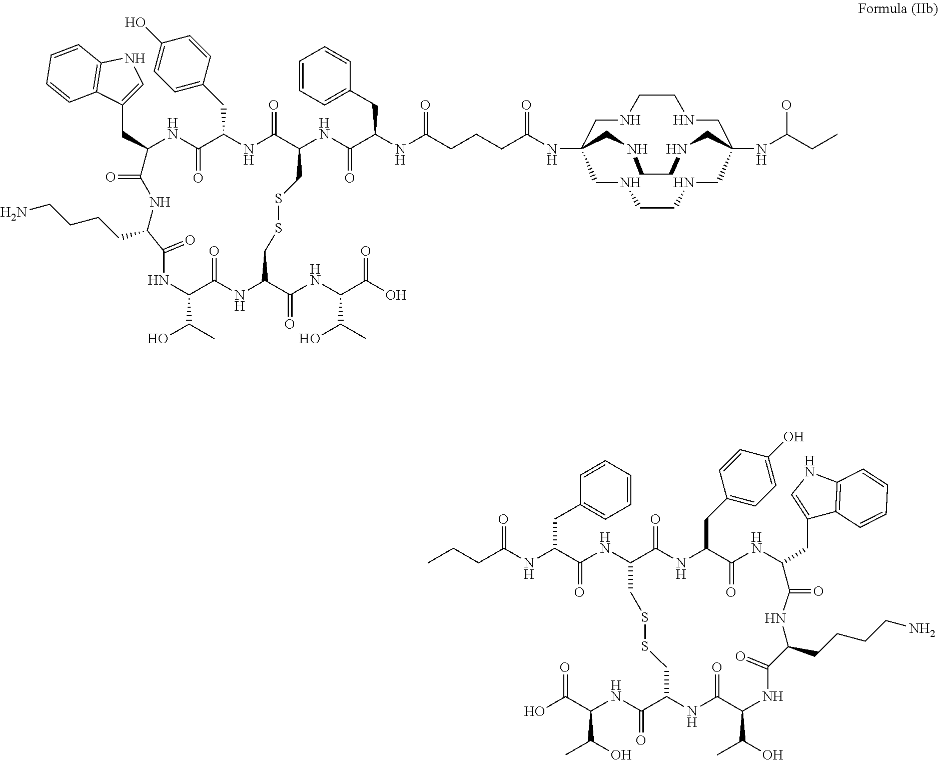
or a pharmaceutically acceptable salt, complex, isomer, solvate or prodrug thereof.

7. A compound according to any one of claims 1 to 6, wherein the metal chelator is complexed with an ion of metal selected from the consisting of Cu, Ga, Lu, F, Tc, In, Zr, Y, Rb, Ac, Rd, Re, Sm, Lu, Sc, Zr, Y, Ac, As, Ra and I.

8. A compound according to claim 7, wherein the ion is a radionuclide.

9. A compound according to any one of claims 1 to 8, wherein the compound is complexed with a Cu radionuclide.

10. A compound according to claim 9, wherein the Cu radionuclide is selected from the group consisting of ⁶⁰Cu, ⁶¹Cu, ⁶²Cu, ⁶⁴Cu and ⁶⁷Cu.

11. A composition comprising a compound according to any one of claims 1 to 10 and one or more pharmaceutically acceptable excipients.

12. A method for radioimaging a cancer in a subject in need thereof, the method comprising administering to the subject a compound according to any one of claims 7 to 10.

13. A method according to claim 12, wherein the cancer is characterised by the expression of SSTR2.

14. A method according to claim 12 or 13, wherein the cancer is selected from the group consisting of a neuroendocrine tumour including a carcinoid tumour in the lung, appendix, digestive tract, prostate, thymus or rectum, a pancreatic neuroendocrine tumour, pituitary tumour, renal cell cancer, breast cancer, meningioma, glioma, neuroblastoma, colorectal cancer, pheochromocytoma, medullary thyroid cancer, gastrinoma, insulinoma or non-functioning islet cell tumour, ovarian cancer, head and/or neck cancer, gastric cancer, adrenal cancer, brain cancer, thyroid cancer and a hematologic malignancy such as lymphoma or leukaemia.

15. A method for the treatment of a cancer in a subject in need thereof, the method comprising administering to the subject a compound according to any one of claims 7 to 10.

16. A method according to claim 15, wherein the cancer is characterised by the expression of SSTR2.

17. A method according to claim 15 or 16 wherein the cancer is selected from the group consisting of a neuroendocrine tumour including a carcinoid tumour in the lung, appendix, digestive tract, prostate, thymus or rectum, a pancreatic neuroendocrine tumour, pituitary tumour, renal cell cancer, breast cancer, meningioma, glioma, neuroblastoma, colorectal cancer, pheochromocytoma, medullary thyroid cancer, gastrinoma, insulinoma or non-functioning islet cell tumour, ovarian cancer, head and/or neck cancer, gastric cancer, adrenal cancer, brain cancer, thyroid cancer and a hematologic malignancy such as lymphoma or leukaemia.

18. Use of a compound according to any one of claims 1 to 8 in the manufacture of a medicament for radioimaging a cancer.

19. Use according to claim 14, wherein the cancer is characterised by the expression of SSTR2.

20. Use according to claim 14 or 15, wherein the cancer is selected from the group consisting of a neuroendocrine tumour including a carcinoid tumour in the lung, appendix, digestive tract, prostate, thymus or rectum, a pancreatic neuroendocrine tumour, pituitary tumour, renal cell cancer, breast cancer, meningioma, glioma, neuroblastoma, colorectal cancer, pheochromocytoma, medullary thyroid cancer, gastrinoma, insulinoma or non-functioning islet cell tumour, ovarian cancer, head and/or neck cancer, gastric cancer, adrenal cancer, brain cancer, thyroid cancer and a hematologic malignancy such as lymphoma or leukaemia.

21. Use of a compound according to any one of claims 1 to 6 in the manufacture of a medicament for treating a cancer.

22. Use according to claim 19, wherein the cancer is characterised by the expression of SSTR2.

23. Use according to claim 19 or 20, wherein the cancer is selected from the group consisting of a neuroendocrine tumour including a carcinoid tumour in the lung, appendix, digestive tract, prostate, thymus or rectum, a pancreatic neuroendocrine tumour, pituitary tumour, renal cell cancer, breast cancer, meningioma, glioma, neuroblastoma, colorectal cancer, pheochromocytoma, medullary thyroid cancer, gastrinoma, insulinoma or non-functioning islet cell tumour, ovarian cancer, head and/or neck cancer, gastric cancer, adrenal cancer, brain cancer, thyroid cancer and a hematologic malignancy such as lymphoma or leukaemia.
